(19) United States
(12) Patent Application Publication
(10) Pub. No.: US 2010/0087012 A1
(43) Pub. Date: Apr. 8, 2010

(54) Sample Collector and Processor

(75) Inventor: Alexander Shenderov, Raleigh, NC (US)

Correspondence Address: ADVANCED LIQUID LOGIC, INC.;C/O WARD AND SMITH, P.A., 1001 COLLEGE COURT, P.O. BOX 867, NEW BERN, NC, 28563-0867, US

(73) Assignee: ADVANCED LIQUID LOGIC, INC., Research Triangle Park, NC (US)

(21) Appl. No.: 12/596,902

(22) PCT Filed: Apr. 23, 2008
(86) PCT No.: PCT/US08/61295

§ 371 Date: Oct. 21, 2009

Related U.S. Application Data
(60) Provisional application No. 60/913,400, filed on Apr. 23, 2007.

(51) Int. Cl. G01N 33/543; B01L 3/00; G01N 1/28

(52) U.S. Cl. 436/518; 422/99; 422/67; 422/63; 436/177; 901/50

(57) ABSTRACT

An aerosol sample collector with an air flow path comprising: (i) at a first segment thereof, a particle charging device, and (i) at a second segment thereof, deflection plates configured to focus particles of a desired charge into a preselected cross-section of the air flow path. The air flow path also includes charged substrates arranged at an outflow portion of the air flow path to collect charged particles on a collection surface of the charged substrates; and an exit path for flowing particles not in the preselected cross-section of the air flow path away from the charged substrates. Related methods are also provided.

### BACKGROUND

As methods of analysis require smaller and smaller input samples, there is a need in the art for approaches to preparing small samples from large samples sources. One such issue is the preparation of small samples including aerosols from larger fluid samples.

Droplet actuators are used to conduct a wide variety of droplet operations. A droplet actuator typically includes a substrate comprising electrodes arranged for conducting droplet operations. The droplet actuator may also include a top plate separated from a droplet operations surface of the substrate to form a gap in which droplet operations may be effected The top plate may also include electrodes for conducting droplet operations. The space is typically filled with a filler fluid that is immiscible with the fluid that is to be manipulated on the droplet actuator. Surfaces exposed to the space are typically hydrophobic. There is a need in the art for approaches to using small-volume droplet actuator based assays to analyze analytes from large volume air samples.

### SUMMARY OF THE INVENTION

The invention provides an aerosol sample collector. The collector may include an air flow path. The air flow path may include at a first air flow segment thereof, a particle charging device configured for charging particles flowing through the path. The air flow path may include at a second segment thereof, deflection plates configured to focus particles of a predetermined charge into a predetermined cross-section of the air flow path, such as a centrally located region within a cross-section of the air flow path.

The collector may further include one or more charged substrates arranged at an outflow portion of the air flow path to collect charged particles on a collection surface of the charged substrates. In certain embodiments, the collection surface is hydrophobic in character. For example, the collection surface may include a hydrophobic coating.

Further, in some embodiments, the collector may include an exit path for flowing particles not in the preselected cross-section of the air flow path away from the charged substrates. The exit path may, for example, be formed by the use of louvers.

In some cases, the collection surface and/or a surface separated from the collection surface by a gap comprises a droplet operations surface of a droplet actuator. Thus, for example, the collection surface may include droplet operations electrodes associated therewith for transporting a droplet across the droplet operations surface.

In other embodiments, the invention provides a system that includes the aerosol sample collector of claim including droplet operations electrodes associated with the collection surface and configured for conducting one or more droplet operations thereon, causing the collection surface to function as a droplet operations surface. The system may be controlled by a computer processor. In some embodiments, the computer is programmed to transport a droplet across the droplet operations surface to collect particles from the collection surface. The system may also include one or more reagent droplets on the droplet operations surface and may be used to conduct droplet operations (e.g., pre-programmed or based on real time instructions from with a user) to execute an assay protocol using the sample droplet.

In a related embodiment, a robotic means is provided for moving a droplet across the sample collection surface to collect particles from the collection surface, yielding a sample droplet comprising one or more particles.

The invention also includes a method of collecting particles, such as aerosol particles. The method generally includes (a) flowing air along a gaseous source material comprising the particles along a flow path; (b) charging particles in the gaseous source material; (c) using deflection plates appropriately positioned to focus the particles into a predetermined cross-section of the flow path; and (d) collecting the particles onto a charged substrate while flowing gaseous source material not in the predetermined cross-section away from the charged substrate. In some embodiments, the collection surface is hydrophobic in character. Gaseous source material not in the predetermined cross-section may be flowed away from the charged substrate by, for example, making use of appropriately arranged louvers.

The method may further include transporting a droplet across the surface of the collection substrate. In this manner, particles may be collected into the droplet, thereby yielding a sample droplet comprising one or more of the particles.

In some embodiments, the sample droplet may be provided onto a droplet operations surface of a droplet actuator, e.g., manually or by making use of electrode mediated transport. The droplet actuator may be employed for execution of one or more steps of a droplet based assay protocol using the droplet.

In some cases, the droplet used to collect particles may include one or more beads and/or may be combined with another droplet including one or more beads. For example, beads may be used having affinity for a target particle and/or affinity for one or more substances associated with the target particle, e.g., multiple sets of beads, each set having affinity for a different target may be used; further, single beads having affinity for multiple targets may also or alternatively be used. The method may also include transporting the droplet to a droplet operations surface of a droplet actuator for execution of one or more steps of a droplet based assay protocol using the droplet. As an example, the one or more steps may comprise steps of a bead washing protocol. The transporting and/or other steps may be mediated by a droplet actuator, manually, and/or by robotic means.

In some cases, the collection surface is also at least a portion of a droplet operations surface of a droplet actuator, and/or the collection surface is also at least a portion of a top substrate of a droplet actuator and is separated by a gap from a droplet operations surface of a droplet actuator.

The method may also include executing droplet operations to effect one or more steps of an assay protocol on the droplet operations surface using the sample droplet. For example, the method may include providing a reagent droplet on the collection surface and executing droplet operations to effect one or more steps of an assay protocol on the collection surface using the sample droplet and the at least one reagent droplet.

In certain embodiments, the method includes providing at least one reagent droplet on the droplet operations surface and executing droplet operations to effect one or more steps of an assay protocol on the droplet operations surface using the sample droplet and the at least one reagent droplet.

In various embodiments one or more of the droplet operations may be electrode mediated. For example one or more of the droplet operations may be electrowetting mediated and/or dielectrophoresis-mediated.

These and other embodiments will be apparent from the ensuing description of the invention, the figures, and the claims.

### BRIEF DESCRIPTION OF THE FIGURES

FIG. 1 shows an aerosol sample collector illustrating (a) collection of particles on a plate; and (b) collection from plate into a droplet.

FIG. 2 shows an electrowetting-based collection mechanism.

### DEFINITIONS

As used herein, the following terms have the meanings indicated.

“Activate” with reference to one or more electrodes means effecting a change in the electrical state of the one or more electrodes which results in a droplet operation.

“Bead,” with respect to beads on a droplet actuator, means any bead or particle that is capable of interacting with a droplet on or in proximity with a droplet actuator. Beads may be any of a wide variety of shapes, such as spherical, generally spherical, egg shaped, disc shaped, cubical and other three dimensional shapes. The bead may, for example, be capable of being transported in a droplet on a droplet actuator; configured with respect to a droplet actuator in a manner which permits a droplet on the droplet actuator to be brought into contact with the bead, on the droplet actuator and/or off the droplet actuator. Beads may be manufactured using a wide variety of materials, including for example, resins, and polymers. The beads may be any suitable size, including for example, microbeads, microparticles, nanobeads and nanoparticles. In some cases, beads are magnetically responsive; in other cases beads are not significantly magnetically responsive. For magnetically responsive beads, the magnetically responsive material may constitute substantially all of a bead or one component only of a bead. The remainder of the bead may include, among other things, polymeric material, coatings, and moieties which permit attachment of an assay reagent. Examples of suitable magnetically responsive beads are described in U.S. Patent Publication No. 2005-0260686, entitled, “Multiplex flow assays preferably with magnetic particles as solid phase,” published on Nov. 24, 2005, the entire disclosure of which is incorporated herein by reference for its teaching concerning magnetically responsive materials and beads. It should also be noted that various droplet operations described herein which can be conducted using beads can also be conducted using biological cells.

“Droplet,” with reference to droplet operations, means a volume of liquid on a droplet actuator surface which is at least partially bounded by filler fluid (which may, for example, be oil or air). For example, a droplet may be completely surrounded by filler fluid or may be bounded by filler fluid and one or more surfaces of the droplet actuator. Droplets may take a wide variety of shapes; nonlimiting examples include generally disc shaped, slug shaped, truncated sphere, ellipsoid, spherical, partially compressed sphere, hemispherical, ovoid, cylindrical, and various shapes formed during droplet operations, such as merging or splitting or formed as a result of contact of such shapes with one or more surfaces of a droplet actuator.

“Droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to size of the resulting droplets (i.e., the size of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading.

“Immobilize” with respect to magnetically responsive beads, means that the beads are substantially restrained in position in a droplet or in filler fluid on a droplet actuator. For example, in one embodiment, immobilized beads are sufficiently restrained in position to permit execution of a spiltting operation on a droplet, yielding one droplet with substantially all of the beads and one droplet substantially lacking in the beads.

“Magnetically responsive” means responsive to a magnetic field. Examples of magnetically responsive materials include paramagnetic materials, ferromagnetic materials, ferrimagnetic materials, and metamagnetic materials. Examples of suitable paramagnetic materials include iron, nickel, and cobalt, as well as metal oxides, such as Fe₃0₄, BaFe₁₂O₁₉, CoO, NiO, Mn₂O₃, Cr₂O₃, and CoMnP.

“Washing” with respect to washing a magnetically responsive bead means reducing the amount of one or more substances in contact with the magnetically responsive bead or exposed to the magnetically responsive bead from a droplet in contact with the magnetically responsive bead. The reduction in the amount of the substance may be partial, substantially complete, or even complete. The substance may be any of a wide variety of substances; examples include target substances for further analysis, and unwanted substances, such as components of a sample, contaminants, and/or excess reagent. In some embodiments, a washing operation begins with a starting droplet in contact with a magnetically responsive bead, where the droplet includes an initial total amount of a substance. The washing operation may proceed using a variety of droplet operations. The washing operation may yield a droplet including the magnetically responsive bead, where the droplet has a total amount of the substance which is less than the initial amount of the substance. Other embodiments are described elsewhere herein, and still others will be immediately apparent in view of the present disclosure.

The terms “top” and “bottom” are used throughout the description with reference to the top and bottom substrates of the droplet actuator for convenience only, since the droplet actuator is functional regardless of its position in space.

When a given component such as a layer, region or substrate is referred to herein as being disposed or formed “on” another component, that given component can be directly on the other component or, alternatively, intervening components (for example, one or more coatings, layers, interlayers, electrodes or contacts) can also be present. It will be further understood that the terms “disposed on” and “formed on” are used interchangeably to describe how a given component is positioned or situated in relation to another component. Hence, the terms “disposed on” and “formed on” are not intended to introduce any limitations relating to particular methods of material transport, deposition, or fabrication.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface.

When a droplet is described as being “on” or “loaded on” a droplet actuator, it should be understood that the droplet is arranged on the droplet actuator in a manner which facilitates using the droplet actuator to conduct droplet operations on the droplet, the droplet is arranged on the droplet actuator in a manner which facilitates sensing of a property of or a signal from the droplet, and/or the droplet has been subjected to a droplet operation on the droplet actuator.

### DETAILED DESCRIPTION

Disclosed are a series of devices for concentrating aerosol particles from intake gas into small liquid volumes that are primarily intended for analysis of aerosol particles in air or other gases. The devices comprise: an air inlet; solid surface(s) for collection of particles; a water droplet dispenser; and means of perambulating the droplet(s) formed by said dispenser across said solid surface(s). The devices can also optionally comprise: means of aerosol concentration; means of particle size separation; means of electrically charging aerosol particles; and/or means of forcing air through an air path, at least partially formed by said air inlet and said solid surface.

### 5.1 Devices For Concentrating Aerosol Particles

In one embodiment, the sample collector comprises an air inlet with a passthrough corona discharge arrangement; an inlet air duct with parallel electrodes on opposite sides; one or more louvers subdividing the airflow into multiple ducts; at least one system of parallel plates forming some of the walls of said ducts and/or positioned parallel to said walls; means of applying controlled charge to said plates, such as known high-voltage circuits; and means of actuating the airflow, such as fans, vacuum pumps, and/or Venturi tubes. It further comprises a droplet dispenser and a mechanical scanning means of moving a needle with a droplet (or multiple needles with droplets) attached to the needle(s) in a pattern over a plate with the droplet touching the plate as it moves over it. The pattern is preferably a meander pattern chosen to cover substantially the entire area of expected electrostatic deposition of aerosol particles, and its pitch should not exceed the width of the droplet footprint on the plate. The area of expected electrostatic deposition of aerosol particles should be made hydrophobic by choosing the appropriate hydrophobic material or applying a hydrophobic coating.

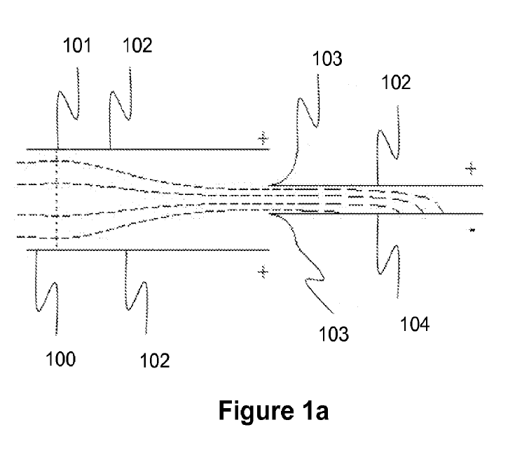

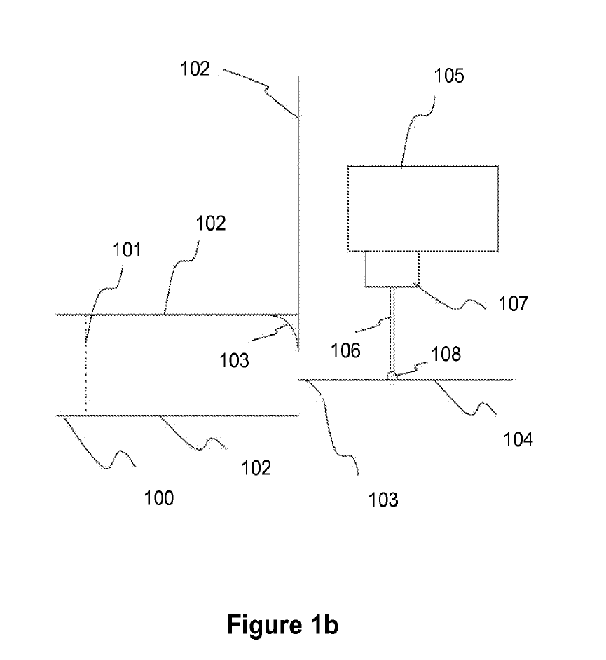

FIG. 1 shows an aerosol sample collector illustrating (a) collection of particles on a plate; and (b) collection from plate into a droplet. This embodiment is operated as follows. During the first phase of operation, shown in FIG. 1(a), airflow is pulled through the air inlet 100 and past the corona charger 101. Aerosol particles in the airstream become charged. Deflection plates 102 on both sides of the first air duct are charged to a high voltage of the same polarity as the particle charge, thus focusing the particles in the center of the airstream. Louvers 103 deflect the portion of the airflow containing no particles away from the second air duct, which is formed by another of the deflection plates 102 and a collection plate 104. The collection plate 104, which should be made hydrophobic by an appropriate choice of material, surface coating (or surface treatment), is charged to a high voltage of opposite polarity to that of the particles, so that the particles are electrostatically attracted to the plate 104 and captured there. The airflow is actuated by a fan (not shown) in the airstream deflected by louvers 104, and the airstream in the second air duct is connected to that airflow through a Venturi tube, providing for an appropriate flow-rate ratio.

After completion of a sampling period of a specified duration, a mechanical motion 105 is used to bring needle 106, attached to liquid handling mechanism 107, into proximity with the plate 104. If necessary, some of the louvers 103, plates 102, and other parts may be repositioned to allow sufficient clearance, as shown in FIG. 1(b). Droplet 108 of collection liquid is brought in contact with plate 104, and motion 105 perambulates the droplet across substantially the entire area of particle deposition within the plate 104. After the perambulation is complete, the droplet 108 can be sucked back into the needle 108, or detached from it for further processing.

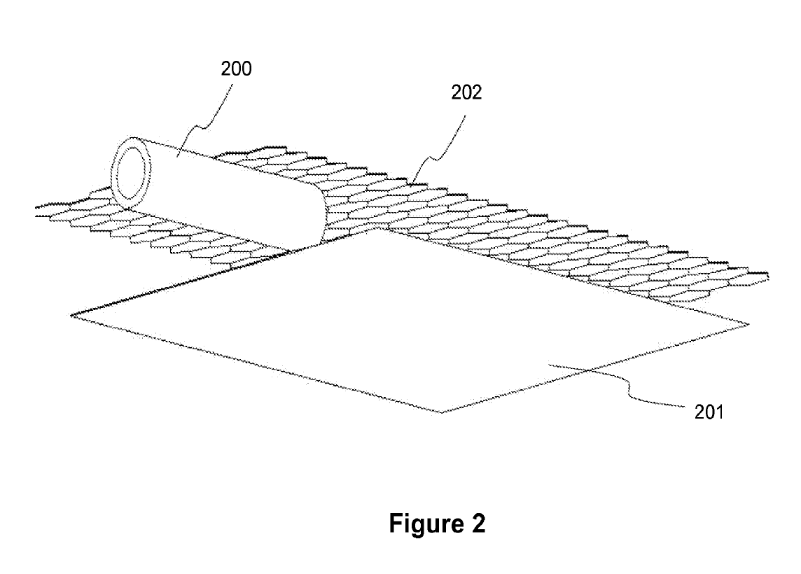

FIG. 2 shows an electrowetting-based collection mechanism. The mechanical scanning means are replaced with a plate carrying electrodes for electrowetting-based actuation of droplets. In a further variant, the electrode-carrying plate is the same as one of the charged plates, and is equipped with the means for controlling the distance from it to the opposing charged plate where the particles are collected.

If electrowetting-based droplet actuation is employed, the device may be operated as follows. The first phase of operation is as described above. For the second phase, collection liquid is presented by the liquid handling mechanism 107 through needle(s) 200 to an electrowetting plate(s). The electrowetting plate(s) carries a pattern of electrodes 201 that can be controlled so as to transfer the droplet from one electrode (or group of electrodes) to the next. The electrodes are covered with a dielectric layer, which should be made hydrophobic by an appropriate choice of material, surface coating, or surface treatment. Electrodes on the electrowetting plate are actuated to effect detachment of the droplet(s) 108 from the needle(s) 106, optionally in conjunction with pulling collection liquid back through the needle(s) 106 by the liquid handling mechanism 107. The droplet(s) are further actuated to effect movement of the droplet(s) along a predetermined path to perambulate the droplet(s) across substantially the entire area of particle deposition within the plate 202.

After the perambulation is complete, the droplet(s) 108 can be sucked back into the needle(s) 106, or detached from the electrowetting plate by another mechanism, such as gravity. If gravity collection of droplets is employed, the active side of the electrowetting plate should be facing down, and the plate should be positioned to create an overhang over the collection plate(s) 202.

In another embodiment, the sample collector comprises: an air inlet; filter, or multiple filters, for collecting aerosol particles; a droplet dispenser and a mechanical scanning means moving a needle with a droplet (or multiple needles with droplets) attached to the needle(s) in a pattern over the filter(s) with the droplet touching the filter(s) as it moves over them. The chosen pattern should cover substantially the entire area of the filter(s), and its pitch should not exceed the width of the droplet footprint on the plate. In a variant of this embodiment, the filters are attached to porous backing material to improve rigidity and flatness. In another variant, the filter(s) themselves are moveable. In a further variant, both the filters and the droplet are moveable in complementary patterns; for example, a filter in the shape of a disc rotates around its axis, and the droplet scans along its radius. The filters should be surface filters, and they should be made hydrophobic by appropriate choice of material, coating, and/or surface treatment.

Optionally, the sample collector disclosed in this invention can also comprise additional modules for controlling and/or measuring airflow and preconcentrating and/or preselecting aerosol particles of certain size ranges, including, but not limited to, cyclones, electrocyclones, virtual impactors, actuated louvers and flowmeters.

### 5.2 Droplet Actuator

For examples of droplet actuator architectures suitable for use with the present invention, see U.S. Pat. No. 6,911,132, entitled “Apparatus for Manipulating Droplets by Electrowetting-Based Techniques,” issued on Jun. 28, 2005 to Pamula et al.; U.S. patent application Ser. No. 11/343,284, entitled “Apparatuses and Methods for Manipulating Droplets on a Printed Circuit Board,” filed on filed on Jan. 30, 2006; U.S. Pat. Nos. 6,773,566, entitled “Electrostatic Actuators for Microfluidics and Methods for Using Same,” issued on Aug. 10, 2004 and U.S. Pat. No. 6,565,727, entitled “Actuators for Microfluidics Without Moving Parts,” issued on Jan. 24, 2000, both to Shenderov et al.; Pollack et al., International Patent Application No. PCT/US 06/47486, entitled “Droplet-Based Biochemistry,” filed on Dec. 11, 2006, the disclosures of which are incorporated herein by reference. Methods of the invention may be executed using droplet actuator systems, e.g., as described in International Patent Application No. PCT/US2007/09379, entitled “Droplet manipulation systems,” filed on May 9, 2007. Examples of droplet actuator techniques for immobilizing magnetic beads and/or non-magnetic beads in the context of bead washing and/or conducting assays are described in the foregoing international patent applications and in Sista, et al., U.S. patent application Ser. Nos. 60/900,653, filed on Feb. 9, 2007, entitled “Immobilization of magnetically-responsive beads during droplet operations”; Sista et al., U.S. patent application Ser. No. 60/969,736, filed on Sep. 4, 2007, entitled “Droplet Actuator Assay Improvements”; and Allen et al., U.S. patent application Ser. No. 60/957,717, filed on Aug. 24, 2007, entitled “Bead washing using physical barriers,” the entire disclosures of which is incorporated herein by reference.

### 5.3 Filler Fluids

The gap will typically be filled with a filler fluid. The filler fluid may, for example, be a low-viscosity oil, such as silicone oil. Other examples of filler fluids are provided in International Patent Application No. PCT/US 06/47486, entitled “Droplet-Based Biochemistry,” filed on Dec. 11, 2006. The filler fluid may be a gas, such as air.

This specification is divided into sections for the convenience of the reader only. Headings should not be construed as limiting of the scope of the invention.

It will be understood that various details of the present invention may be changed without departing from the scope of the present invention. Various aspects of each embodiment described here may be interchanged with various aspects of other embodiments. Furthermore, the foregoing description is for the purpose of illustration only, and not for the purpose of limitation.

I claim:

1. An aerosol sample collector comprising:
(a) an air flow path comprising:
(i) at a first segment thereof, a particle charging device; and
(ii) at a second segment thereof, deflection plates configured to focus particles of a desired charge into a preselected cross-section of the air flow path;
(b) charged substrates arranged at an outflow portion of the air flow path to collect charged particles on a collection surface of the charged substrates;
(c) an exit path for flowing particles not in the preselected cross-section of the air flow path away from the charged substrates.

2. The aerosol sample collector of claim 1 wherein the collection surface is hydrophobic in character.

3. The aerosol sample collector of claim 1 wherein the exit path is created by the use of louvers.

4. The aerosol sample collector of claim 1 wherein the collection surface and/or a surface separated from the collection surface by a gap comprises a droplet operations surface of a droplet actuator.

5. A system comprising:
(a) the aerosol sample collector of claim 4;
(b) a computer processor programmed to control droplet operations on the droplet actuator and further programmed to transport a droplet across the droplet operations surface to collect particles from the collection surface.

6. The system of claim 5:
(a) comprising one or more reagent droplets on the droplet operations surface; and
(b) further programmed to conduct droplet operations to execute an assay protocol using the sample droplet.

7. A system comprising:
(a) the aerosol sample collector of claim 1; and
(b) a robotic means for moving a droplet across the sample collection surface to collect particles from the collection surface, yielding a sample droplet comprising one or more particles.

8. An apparatus comprising:
(a) a substrate comprising a charged collection surface for collecting particles, wherein the charged collection surface comprises droplet operations electrodes associated therewith and serves as a droplet operations surface for droplet operations conducted by the electrodes; and
(b) a means for flowing particle-containing gaseous source material into contact with the collection surface.

9. A method of collecting particles, the method comprising:
(a) flowing air along a gaseous source material comprising the particles along a flow path;
(b) charging particles in the gaseous source material;
(c) using deflection plates appropriately positioned to focus the particles into a predetermined cross-section of the flow path; and
(d) collecting the particles onto a charged substrate while flowing gaseous source material not in the predetermined cross-section away from the charged substrate.

10. The method of claim 9 wherein the collection surface is hydrophobic in character.

11. The method of claim 9 wherein flowing gaseous source material not in the predetermined cross-section away from the charged substrate is accomplished a means comprising the use of louvers.

12. The method of claim 9 further comprising transporting a droplet across the surface of the collection substrate in order to collect particles into the droplet, thereby yielding a sample droplet comprising one or more of the particles.

13. The method of claim 12 further comprising transporting the droplet to a droplet operations surface of a droplet actuator for execution of one or more steps of a droplet based assay protocol using the droplet.

14. The method of claim 12 wherein the droplet comprises one or more beads having affinity for a target particle and/or affinity for a substance associated with the target particle.

15. The method of claim 14 further comprising transporting the droplet to a droplet operations surface of a droplet actuator for execution of one or more steps of a droplet based assay protocol using the droplet.

16. The method of claim 14 wherein the one or more steps comprise steps of a bead washing protocol.

17. The method of claim 9 wherein the transporting is mediated by a droplet actuator.

18. The method of claim 9 wherein the transporting is mediated by a robotic means.

19. The method of claim 17 wherein:
(a) the collection surface is also at least a portion of a droplet operations surface of a droplet actuator; and/or
(b) the collection surface is also at least a portion of a top substrate of a droplet actuator and is separated by a gap from a droplet operations surface of a droplet actuator.

20. The method of claim 17 further comprising executing droplet operations to effect one or more steps of an assay protocol on the droplet operations surface using the sample droplet.

21. The method of claim 17 further comprising providing a reagent droplet on the collection surface and executing droplet operations to effect one or more steps of an assay protocol on the collection surface using the sample droplet and the at least one reagent droplet.

22. The method of claim 21 wherein one or more of the droplet operations is electrode mediated.

23. The method of claim 21 wherein one or more of the droplet operations is electrowetting mediated.

24. The method of claim 21 wherein one or more of the droplet operations is dielectrophoresis-mediated.

25. The method of claim 19 further comprising providing at least one reagent droplet on the droplet operations surface and executing droplet operations to effect one or more steps of an assay protocol on the droplet operations surface using the sample droplet and the at least one reagent droplet.

26. The method of claim 9 wherein the transporting is controlled by a computer.
